(19) United States
(12) Patent Application Publication
(10) Pub. No.: US 2026/0091057 A1
(43) Pub. Date: Apr. 2, 2026

(54) SUPPLEMENTS FOR FERTILITY HEALTH

(71) Applicant: Hannah Life Technologies PTE, Ltd., Singapore (SG)

(72) Inventors: Prusothman M. SINA RAJA, Singapore (SG); Chee Keong TEE, Singapore (SG); Zong Yuan XU, Signapore (SG); Jaron Hong Yi LEE, Singapore (SG); Bao Qi TAN, Singapore (SG)

(73) Assignee: Hannah Life Technologies PTE, Ltd., Singapore (SG)

(21) Appl. No.: 19/341,322

(22) Filed: Sep. 26, 2025

Related U.S. Application Data
(60) Provisional application No. 63/699,861, filed on Sep. 27, 2024.

(51) Int. Cl. A61K 33/30; A61K 31/01; A61K 31/07; A61K 31/122; A61K 31/355; A61K 31/375; A61K 31/519; A61K 31/593; A61K 31/7004; A61K 31/714; A61K 33/04; A61K 33/06; A61K 33/18; A61K 33/26

(52) U.S. Cl. CPC A61K 33/30; A61K 31/01; A61K 31/07; A61K 31/122; A61K 31/355; A61K 31/375; A61K 31/519; A61K 31/593; A61K 31/7004; A61K 31/714; A61K 33/04; A61K 33/06; A61K 33/18; A61K 33/26

(57) ABSTRACT

Disclosed are nutritional supplements for administration to women and to men. A nutritional supplement for administration to women comprises: Vitamin A, Vitamin D, folic acid, iron, iodine, magnesium, zinc, selenium, CoQ10, myo-inositol, D-chiro-inositol, and optionally Vitamin B12. A nutritional supplement for administration to men comprises Vitamin C, Vitamin E, folic acid, zinc, selenium, lycopene, CoQ10; and myo-inositol.

### BACKGROUND

Infertility is a common condition that affects as many as one in six couples trying to conceive. Women and men trying to conceive may wish to supplement their diets with additional nutrients that have been shown to support fertility health. Such a supplement is tailored to the needs of women or men and may be provided as a daily-use oral supplement.

### SUMMARY

Disclosed are nutritional supplements for administration to women and to men. In one embodiment, a nutritional supplement for administration to women comprises: Vitamin A, Vitamin D, folic acid, iron, iodine, magnesium, zinc, selenium, CoQ10, myo-inositol, D-chiro-inositol, and optionally Vitamin B12. In one embodiment, a nutritional supplement for administration to men comprises Vitamin C, Vitamin E, folic acid, zinc, selenium, lycopene, CoQ10; and myo-inositol.

### DETAILED DESCRIPTION

In one embodiment, a nutritional supplement for administration to women comprises: Vitamin A, Vitamin D, folic acid, iron, iodine, magnesium, zinc, selenium, CoQ10, myo-inositol, D-chiro-inositol, and optionally Vitamin B12. In one embodiment, a nutritional supplement for administration to women comprises: Vitamin A, Vitamin D, folic acid, iron, iodine, magnesium, zinc, selenium, CoQ10, myo-inositol, D-chiro-inositol, and Vitamin B12. In one embodiment, a nutritional supplement for administration to women consists essentially of: Vitamin A, Vitamin D, folic acid, iron, iodine, magnesium, zinc, selenium, CoQ10, myo-inositol, D-chiro-inositol, and optionally Vitamin B12. In one embodiment, a nutritional supplement for administration to women consists essentially of: Vitamin A, Vitamin D, folic acid, iron, iodine, magnesium, zinc, selenium, CoQ10, myo-inositol, D-chiro-inositol, and Vitamin B12.

In one embodiment, a nutritional supplement for administration to men comprises: Vitamin C, Vitamin E, folic acid, zinc, selenium, lycopene, CoQ10; and myo-inositol. In one embodiment, a nutritional supplement for administration to men consists essentially of: Vitamin C, Vitamin E, folic acid, zinc, selenium, lycopene, CoQ10; and myo-inositol

The disclosed nutritional supplements may be formulated such that they are free of heavy metals. In one embodiment, the nutritional supplement for administration to women does not comprise any of arsenic, cadmium, lead, or mercury. In one embodiment, the nutritional supplement for administration to men does not comprise any of arsenic, cadmium, lead, or mercury.

The disclosed nutritional supplements may be formulated using excipients known to the skilled person. For example, the nutritional supplements may comprise a binder such as microcrystalline cellulose, or an anti-adherent such as magnesium stearate or silica. Alternatively or additionally the nutritional supplements may comprise flavorings or colors.

The nutritional supplements may be in the form of tablets or other forms known to the skilled person, e.g. capsules, soft gels, or gummies. In one embodiment, the nutritional supplements are in the form of capsules having a 00 capsule size.

The amounts of the different components in the nutritional supplements may be selected to maximize fertility benefits while ensuring regulatory compliance. Ensuring regulatory compliance may include following established upper intake limits for many nutrients which regulatory bodies enforce to prevent potential harm.

The amounts recited for each component are the mass of the component that is present in the daily dose of the supplement. In one embodiment, the daily dose is a single article such as one tablet, one capsule, one soft gel, or one gummy. In an alternative embodiment, the daily dose is split across several articles such that the desired amounts are present in several articles such as two or more tablets, two or more capsules, two or more soft gels, or two or more gummies. In one embodiment, the daily dose is split between three articles, such as three tablets, three capsules, three soft gels or three gummies.

In one embodiment, the nutritional supplement for administration to women comprises Vitamin A. It has been reported that greater intake of Vitamin A may be associated with decreased risk of female infertility. In one embodiment, the amount of Vitamin A is from 0.74 to 3 mg. In another embodiment, the amount of Vitamin A is from 0.74 to 1 mg.

In one embodiment, the nutritional supplement for administration to women comprises Vitamin D. It has been reported that Vitamin D can improve the menstrual cycle and folliculogenesis. In one embodiment, the amount of Vitamin D is from 0.025 to 0.15 mg. In another embodiment, the amount of Vitamin D is from 0.025 to 0.1 mg.

In one embodiment, the nutritional supplement for administration to women comprises folic acid. The folic acid may be supplied as a glucosamine salt of (6S)-5-methyltetrahydrofolic acid. It has been reported that folic acid can increase luteal progesterone levels, can reduce the risk of anovulation, and can increase the absorption rate of synthetic folate. In one embodiment, the amount of folic acid is from 0.4 to 1 mg. In another embodiment the amount of folic acid is from 0.8 to 1 mg. If the folic acid is provided as a salt of folic acid, the amount includes the mass of the counterion, e.g. includes the mass of the glucosamine counterion.

In one embodiment, the nutritional supplement for administration to women comprises iron. It has been reported that there may be a correlation between low iron levels and unexplained infertility. The iron is suitably present as an iron salt such as ferrous fumarate. In one embodiment, the amount of iron (measured as mass of iron salt if supplied as a salt) is from 15 to 45 mg. In another embodiment, the amount of iron is from 15 to 20 mg.

In one embodiment, the nutritional supplement for administration to women comprises iodine. It has been reported that iodine, which is essential for normal thyroid hormone synthesis and release, can indirectly promote ovulation. The iodine may be present as an iodide salt, such as potassium iodide. In one embodiment, the amount of iodine (measured as mass of iodide salt if supplied as a salt) is from 0.15 to 1.1 mg. In another embodiment, the amount of iodine is from 0.15 to 0.35 mg.

In one embodiment, the nutritional supplement for administration to women comprises magnesium. It has been reported that magnesium deficiency may increase the risk of infertility, the risk of miscarriage, the risk of pre-term birth, and the risk of low birthweight babies. The magnesium is suitably present as a magnesium salt such as magnesium citrate or magnesium oxide. In one embodiment, the amount of magnesium (measured as mass of the magnesium salt if supplied as a salt) is from 100 to 350 mg. In another embodiment, the amount of magnesium is from 100 to 250 mg.

In one embodiment, the nutritional supplement for administration to women comprises zinc. It has been reported that an adequate supply of zinc is necessary for an oocyte to form from a fertilization-competent egg. The zinc is suitably present as a zinc salt such as zinc sulphate, zinc acetate, zinc citrate or zinc gluconate. In one embodiment, the amount of zinc (measured as mass of zinc salt if supplied as a salt) is from 8 to 40 mg. In another embodiment, the amount of zinc is from 8 to 14 mg.

In one embodiment, the nutritional supplement for administration to women comprises selenium. It has been reported that low selenium levels could lead to greater risk of subfertility. In one embodiment, the amount of selenium is from 0.055 to 0.4 mg. In another embodiment, the amount of selenium is from 0.055 to 0.2 mg.

In one embodiment, the nutritional supplement for administration to women comprises CoQ10. It has been reported that CoQ10 increases the probability of pregnancy, possibly by improving oocyte maturation or by reducing cellular oxidative stress. In one embodiment, the amount of CoQ10 is from 150 to 600 mg. In another embodiment, the amount of CoQ10 is from 150 to 200 mg.

In one embodiment, the nutritional supplement for administration to women comprises myo-inositol. It has been reported that myo-inositol plays a role in hormone regulation, may reduce insulin resistance and may induce ovulation in women with polycystic ovary syndrome. In one embodiment, the amount of myo-inositol is from 200 to 4000 mg. In another embodiment, the amount of myo-inositol is from 500 to 1000 mg.

In one embodiment, the nutritional supplement for administration to women comprises D-chiro-inositol. D-chiro-inositol is a stereoisomer of myo-inositol. It has been reported that D-chiro-inositol plays a role in insulin signaling. In one embodiment, the amount of D-chiro-inositol is such that the ratio of myo-inositol to D-chiro-inositol is between 60:1 and 20:1, or is between 50:1 and 30:1, or is about 40:1, or is 40:1. This ratio is both a weight ratio and a molar ratio because the mass of one mole of myo-inositol is the same as the mass of one mole of D-chiro-inositol; they are stereoisomers. This ratio of myo-inositol to D-chiro-inositol is considered to mimic the naturally occurring ratio in a woman's body. Such a ratio may support the production of healthy reproductive hormones and may support cycle regularity. It may also support improved egg quality.

In one embodiment, the nutritional supplement for administration to women optionally comprises Vitamin B12. In one embodiment, the amount of Vitamin B12 is from 0.0024 to 0.6 mg. In another embodiment, the amount of Vitamin B12 is from 0.0024 to 0.01 mg.

In one embodiment, the nutritional supplement for administration to men comprises Vitamin C. It has been reported that Vitamin C may lead to decreased oxidative damage to sperm DNA. In one embodiment, the amount of Vitamin C is from 100 mg to 2000 mg. In another embodiment, the amount of Vitamin C is from 100 mg to 200 mg.

In one embodiment, the nutritional supplement for administration to men comprises Vitamin E. It has been reported that Vitamin E may protect sperm from oxidative stress and damage. In one embodiment the amount of Vitamin E is from 268 to 1000 mg. In another embodiment, the amount of Vitamin E is from 268 to 350 mg.

In one embodiment, the nutritional supplement for administration to men comprises folic acid. The folic acid may be supplied as a glucosamine salt of (6S)-5-methyltetrahydrofolic acid. It has been reported that folic acid may reduce sperm DNA fragmentation and may diminish sperm damage. In one embodiment, the amount of folic acid is from 0.5 to 1 mg. In another embodiment, the amount of folic acid is from 0.8 to 1 mg. If the folic acid is provided as a salt of folic acid, the amount includes the mass of the counterion, e.g. includes the mass of the glucosamine counterion.

In one embodiment, the nutritional supplement for administration to men comprises zinc. It has been reported that zinc may prevent impaired spermatogenesis. The zinc is suitably present as a zinc salt such as zinc sulphate, zinc acetate, zinc citrate or zinc gluconate. In one embodiment, the amount of zinc (measured as mass of zinc salt if supplied as a salt) is from 15 to 40 mg. In another embodiment, the amount of zinc is from 15 to 25 mg.

In one embodiment, the nutritional supplement for administration to men comprises selenium. It has been reported that selenium may be linked with improved sperm motility and morphology. In one embodiment, the amount of selenium is from 0.05 to 0.4 mg. In another embodiment, the amount of selenium is from 0.05 to 0.2 mg.

In one embodiment, the nutritional supplement for administration to men comprises lycopene. It has been reported the lycopene supplements can lead to improved sperm concentration and motility. In one embodiment, the amount of lycopene is from 2 to 45 mg. In another embodiment, the amount of lycopene is from 2 to 8 mg.

In one embodiment, the nutritional supplement for administration to men comprises CoQ10. It has been reported that CoQ10 supplementation may protect sperm from reactive oxygen species, leading to improved sperm count. In one embodiment, the amount of CoQ10 is from 30 to 200 mg. In another embodiment, the amount of CoQ10 is from 150 to 200 mg.

In one embodiment, the nutritional supplement for administration to men comprises myo-inositol. It has been reported that myo-inositol supports mitochondrial function, which is crucial for energy production in sperm cells. In one embodiment, the amount of myo-inositol is from 500 to 1000 mg. In another embodiment, the amount of myo-inositol is from 800 to 1000 mg.

In one embodiment, the nutritional supplement for administration to women comprises from 0.74 to 3 mg of Vitamin A; from 0.025 to 0.15 mg of Vitamin D; from 0.4 to 1 mg of folic acid; from 15 to 45 mg of iron; from 0.15 to 1.1 mg of iodine; from 100 to 350 mg of magnesium; from 8 to 40 mg of zinc; from 0.06 to 0.4 mg of selenium; from 150 to 600 mg of CoQ10; from 200 to 4000 mg of myo-inositol; and an amount of D-chiro-inositol such that the molar ratio of myo-inositol to D-chiro-inositol is between 60:1 and 20:1.

In another embodiment, the nutritional supplement for administration to women comprises from 0.74 to 3 mg of Vitamin A; from 0.025 to 0.15 mg of Vitamin D; from 0.4 to 1 mg of folic acid; from 15 to 45 mg of iron; from 0.15 to 1.1 mg of iodine; from 100 to 350 mg of magnesium; from 8 to 40 mg of zinc; from 0.06 to 0.4 mg of selenium; from 150 to 600 mg of CoQ10; from 200 to 4000 mg of myo-inositol; from 0.0024 to 0.6 mg of Vitamin B12; and an amount of D-chiro-inositol such that the molar ratio of myo-inositol to D-chiro-inositol is between 60:1 and 20:1.

In another embodiment, the nutritional supplement for administration to women comprises from 0.74 to 1 mg of Vitamin A; from 0.025 to 0.1 mg of Vitamin D; from 0.8 to 1 mg of folic acid; from 15 to 20 mg of iron; from 0.15 to 0.35 mg of iodine; from 100 to 250 mg of magnesium; from 8 to 14 mg of zinc; from 0.06 to 0.2 mg of selenium; from 150 to 200 mg of CoQ10; from 500 to 1000 mg of myo-inositol; and an amount of D-chiro-inositol such that the molar ratio of myo-inositol to D-chiro-inositol is between 50:1 and 30:1.

In another embodiment, the nutritional supplement for administration to women comprises from 0.74 to 1 mg of Vitamin A; from 0.025 to 0.1 mg of Vitamin D; from 0.8 to 1 mg of folic acid; from 15 to 20 mg of iron; from 0.15 to 0.35 mg of iodine; from 100 to 250 mg of magnesium; from 8 to 14 mg of zinc; from 0.06 to 0.2 mg of selenium; from 150 to 200 mg of CoQ10; from 500 to 1000 mg of myo-inositol; from 0.0024 to 0.01 mg of Vitamin B12; and an amount of D-chiro-inositol such that the molar ratio of myo-inositol to D-chiro-inositol is between 50:1 and 30:1.

In one embodiment, the nutritional supplement for administration to men comprises from 100 mg to 2000 mg of Vitamin C; from 268 to 1000 mg of Vitamin E; from 0.5 to 1 mg of folic acid; from 15 to 40 mg of zinc; from 0.05 to 0.4 mg of selenium; from 2 to 45 mg of lycopene; from 30 to 200 mg of CoQ10 is from 150 to 200 mg; and from 500 to 1000 mg of myo-inositol.

In another embodiment, the nutritional supplement for administration to men comprises from 100 mg to 200 mg of Vitamin C; from 268 to 350 mg of Vitamin E; from 0.8 to 1 mg of folic acid; from 15 to 25 mg of zinc; from 0.05 to 0.2 mg of selenium; from 2 to 8 mg of lycopene; from 150 to 200 mg of CoQ10 is from 150 to 200 mg; and from 800 to 1000 mg of myo-inositol.

The nutritional supplement may be taken with food and/or water, but it is not necessary to take the supplement with food or water.

The nutritional supplement may be manufactured according to established methods using established techniques,

### EXEMPLARY EMBODIMENTS

An exemplary formulation for a nutritional supplement for administration to women is shown in Table 1. The supplement is provided as a serving size of three capsules.

TABLE 1

| Nutrient | Form | Amount Per Serving | |
|---|---|---|---|
| Vitamin A | Beta-carotene | 740 | μg |
| Vitamin D | Cholecalciferol | 25 | μg |
| Folic Acid | (6S)-5-methyltetrahydrofolic | 800 | μg |
| | acid, glucosamine salt | | |
| Vitamin B12 | Cyanocobalamin | 2.4 | μg |
| Iron | Ferrous fumarate | 15 | mg |
| Iodine | Potassium iodide | 150 | μg |
| Magnesium | Magnesium citrate | 100 | mg |
| Zinc | Zinc sulphate | 11 | mg |
| Selenium | Selenium enriched yeast | 55 | μg |
| CoQ10 | Ubidecarenone | 150 | mg |
| Myo-inositol | — | 1000 | mg |
| D-chiro-inositol | — | 25 | mg |

An exemplary formulation for a nutritional supplement for administration to men is shown in Table 2. The supplement is provided as three capsules.

TABLE 2

| Nutrient | Form | Amount Per Serving | |
|---|---|---|---|
| Vitamin C | Ascorbic Acid | 100 | mg |
| Vitamin E | D-alpha0-tocopherol | 268 | mg |
| Folic Acid | (6S)-5-methyltetrahydrofolic | 500 | μg |
| | acid, glucosamine salt | | |
| Zinc | Zinc sulphate | 15 | mg |
| Selenium | Selenium enriched yeast | 50 | μg |
| Lycopene | — | 6 | mg |
| CoQ10 | Ubidecarenone | 150 | mg |
| Myo-inositol | — | 1000 | mg |

1. A nutritional supplement for administration to women comprising: Vitamin A, Vitamin D, folic acid, iron, iodine, magnesium, zinc, selenium, CoQ10, myo-inositol, and D-chiro-inositol.

2. The nutritional supplement according to claim 1, comprising: from 0.74 to 3 mg of Vitamin A; from 0.025 to 0.15 mg of Vitamin D; from 0.4 to 1 mg of folic acid; from 15 to 45 mg of iron; from 0.15 to 1.1 mg of iodine; from 100 to 350 mg of magnesium; from 8 to 40 mg of zinc; from 0.06 to 0.4 mg of selenium; from 150 to 600 mg of CoQ10; from 200 to 4000 mg of myo-inositol; and an amount of D-chiro-inositol such that the molar ratio of myo-inositol to D-chiro-inositol is between 60:1 and 20:1.

3. The nutritional supplement according to claim 2, further comprising from 0.0024 to 0.6 mg of Vitamin B12.

4. The nutritional supplement according to claim 1, comprising from 0.74 to 1 mg of Vitamin A; from 0.025 to 0.1 mg of Vitamin D; from 0.8 to 1 mg of folic acid; from 15 to 20 mg of iron; from 0.15 to 0.35 mg of iodine; from 100 to 250 mg of magnesium; from 8 to 14 mg of zinc; from 0.06 to 0.2 mg of selenium; from 150 to 200 mg of CoQ10; from 500 to 1000 mg of myo-inositol; and an amount of D-chiro-inositol such that the molar ratio of myo-inositol to D-chiro-inositol is between 50:1 and 30:1.

5. The nutritional supplement according to claim 4, further comprising from 0.0024 to 0.01 mg of Vitamin B12.

6. A nutritional supplement for administration to men comprising: Vitamin C, Vitamin E, folic acid, zinc, selenium, lycopene, CoQ10, and myo-inositol.

7. The nutritional supplement according to claim 6, comprising: from 100 mg to 2000 mg of Vitamin C; from 268 to 1000 mg of Vitamin E; from 0.5 to 1 mg of folic acid; from 15 to 40 mg of zinc; from 0.05 to 0.4 mg of selenium; from 2 to 45 mg of lycopene; from 30 to 200 mg of CoQ10 is from 150 to 200 mg; and from 500 to 1000 mg of myo-inositol.

8. The nutritional supplement according to claim 6, comprising: from 100 mg to 200 mg of Vitamin C; from 268 to 350 mg of Vitamin E; from 0.8 to 1 mg of folic acid; from 15 to 25 mg of zinc; from 0.05 to 0.2 mg of selenium; from 2 to 8 mg of lycopene; from 150 to 200 mg of CoQ10 is from 150 to 200 mg; and from 800 to 1000 mg of myo-inositol.

9. The nutritional supplement according to claim 1, wherein the nutritional supplement is in the form of a single daily article chosen from one tablet, one capsule, one soft gel, or one gummy.

10. The nutritional supplement according to claim 1, wherein a daily dose of the nutritional supplement is provided by more than one article, chosen from tablets, capsules, soft gels, or gummies.

11. The nutritional supplement according to claim 10, wherein the daily dose is provided by three articles.

12. The nutritional supplement according to claim 11, wherein the daily dose is provided by three capsules.

13. The nutritional supplement according to claim 1, further comprising Vitamin B12.

14. The nutritional supplement according to claim 6, wherein the nutritional supplement is in the form of a single daily article chosen from one tablet, one capsule, one soft gel, or one gummy.

15. The nutritional supplement according to claim 6, wherein a daily dose of the nutritional supplement is provided by more than one article, chosen from tablets, capsules, soft gels, or gummies.

16. The nutritional supplement according to claim 15, wherein the daily dose is provided by three articles.

17. The nutritional supplement according to claim 16, wherein the daily dose is provided by three capsules.
